(19) United States
(12) Patent Application Publication
(10) Pub. No.: US 2012/0035507 A1
(43) Pub. Date: Feb. 9, 2012

(54) Device and method for measuring anatomic geometries

(76) Inventors: Ivan George, Columbia, MD (US); Gyusung Lee, Lutherville, MD (US); Adrian Park, Crownsville, MD (US)

(21) Appl. No.: 13/136,086

(22) Filed: Jul. 22, 2011

Related U.S. Application Data
(60) Provisional application No. 61/366,551, filed on Jul. 22, 2010.

(51) Int. Cl. A61B 5/107; A61B 1/00

(52) U.S. Cl. 600/587

(57) ABSTRACT

Provided herein is a device and system for measuring an object geometry. The device comprises a probe with contacting and tracking ends and a tracking means and actuation control unit positioned on the probe. The system further comprises a measurement and post processing unit in electronic communication with the tracking means and/or the actuation control unit. Also provided is a method for measuring a geometry of an anatomic object, for example, a hernia. One or more locations of interest on the object are touched directly with the contacting probe end and the location or motion data of the probe end and of the tracking means are tracked and transmitted to the measurement and post processing unit as the point(s) of interest are touched. The data is processed into the geometric measurement and a representative image may be displayed.

### CROSS-REFERENCE TO RELATED APPLICATIONS

This non-provisional patent application claims benefit of provisional patent application U.S. Ser. No. 61/366,551, filed Jul. 22, 2010, now abandoned, the entirety of which is hereby incorporated by reference.

### BACKGROUND OF THE INVENTION

1. Field of the Invention

The present invention relates generally to the fields of biomedical devices and surgery. More specifically, the present invention provides a device and methods for measuring 2^{nd}- and 3^{rd}-dimensional anatomic defects and/or geometries utilizing direct touch and indirect measurements.

2. Description of the Related Art

Current methods of determining location, size or other measurements of anatomic features, particularly for surgical purposes, require the use of measuring devices such as tape measures or other calibrated apparatuses which can be positioned internally along the feature or defect. Measurements are taken by direct visualization or from an image of the feature showing the measuring apparatus in place. While external measurements are easier to obtain, it also is easier for a practitioner to overestimate the size of a defect. Internal measurements requires inserting a measuring apparatus into the patient near the anatomic feature or defect which may be difficult to access and/or visualize.

Methods for measuring anatomic defects have not changed significantly over the last 15 years. For example, U.S. Pat. No. 5,379,754 discloses an apparatus having an elongated handle and a measuring rod which pivots around the handle for ease of placement. The measuring rod has calibrations printed along its top and bottom sides for measurement of an abdominal hernia. The surgeon manipulates the handle to position the measuring rod with respect to the hernia opening to measure the size of opening.

Carbonell and Cobb (Tricks for Laparoscopic Ventral Hernia Repair, Contemporary Surgery, Vol. 63, No. 8, August 2007) disclose that externally measuring a hernia defect often results in a practitioner's overestimating the size. It is recommended that a surgeon introduces a ruler, cut to half-size, via a trocar and uses graspers to manipulate the ruler to measure the defect. If the defect is greater than the length of the ruler, the ruler must be repositioned and the separate measurements summed.

Thus, the inventors recognize that no system, device, instrument or probe known in the art and currently in production have the specific capabilities to provide location by the use of direct touch and indirect measurements with the combination of motion tracking systems and computational analysis systems. Therefore, there is an increased need in the art for a precision device and improved methods of measuring anatomic features or geometries. Particularly, the prior art is deficient in devices, systems and methods utilizing direct touch to measure and image anatomic defects or anatomic geometries in a subject. The present invention fulfills this longstanding need and desire in the art.

### SUMMARY OF THE INVENTION

The present invention is directed to a device for measuring an object geometry. The device comprises a probe having a distal contacting end and a proximal tracking end, means for tracking a location of the contacting end of the probe positioned near the proximal end electronically connected to the contacting end of the probe and an actuation control unit electronically connected with at least the tracking sensor. A related device further comprises a measurement and post processing unit electronically connected with one or both of the tracking sensor or the actuation control unit. Another related device further comprises means for holding the probe that is positioned at the proximal end of the probe.

The present invention also is directed to a method for measuring a geometry of an anatomic defect in a subject. The method generally comprises locating the anatomic defect in the subject and measuring the geometry of the defect via direct touch with the device described herein. A related method comprises directly touching with the contacting end of the probe one or more points of interest on or proximate to the defect in the subject and tracking the location of the one or more points as each is touched. The signals corresponding to location data of the one or more points are transmitted to the measuring and post processing unit and the received data signals are processed to a geometric measurement of the anatomic defect. Another related method further comprises a step of displaying an image of the measured geometry of the defect. In yet another related method, the anatomic defect is a hernia and the method further comprises sizing a mesh based on the measured geometry and repairing the hernia with the mesh.

The present invention is directed further to system for measuring a geometry of an object. The system comprises a probe having a contacting tip at a distal end and a proximal tracking end; said tip configured for direct touch of points of interest on the object and one or more tracking sensors or sensor array affixed at the proximal probe end electronically connected to the probe tip, said one or more tracking sensors or array configured to track and transmit motion of the probe tip and location of the tracking sensor(s) or array itself. A measurement and post processing unit is in electronic communication with the tracking sensor or sensor array where the unit is configured to process location data of the probe tip and tracking sensor(s) and to convert the data to a visual geometry of the object. An actuation control unit is electronically connected with at least the tracking sensor or the measurement and post processing unit where the actuation control unit is configured to respond to operator commands.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1A depicts an instrument for measuring anatomic geometries comprising a hand-held probe and a tracking/computational system for detecting points of interest in the anatomy and measuring a feature thereof. FIG. 1B shows a hernia with points of interest A and B labeled as in FIG. 1A.

FIG. 2 depicts a measuring device with a unitized tracking system.

FIG. 3 depicts another measuring device with a unitized tracking system showing vectors for probe calibration.

FIG. 4 depicts a measuring device adapted for an endoscope and comprising a clip-on tracking sensor.

FIGS. 5A-5J depict line, oval and star templates (FIGS. 5A-5B) and the resultant oval (FIGS. 5C-5F) and star (FIGS. 5G-5J) tracings using probes with an optical tip/tracking sensor (FIGS. 5C-5D, 5G-5H) or an electromagnetic tip/tracking sensor (FIGS. 5E-5F, 5I-5J).

FIGS. 6A-6L depict solid triangular object (FIG. 6A) and a mannequin's hand (FIG. 6B) and the resultant three-dimensional triangular (FIGS. 6C-6F) and hand (FIGS. 6G-6L) surface tracing using probes with an optical tip/tracking sensor (FIGS. 6C-6D, 6G-6I) or an electromagnetic tip/tracking sensor (FIGS. 6E-6F, 6J-6L).

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one.

As used herein “another” or “other” may mean at least a second or more of the same or different claim element or components thereof. Similarly, the word “or” is intended to include “and” unless the context clearly indicates otherwise. “Comprise” means “include.”

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the term “location” refers to the three dimensional coordinates in x, y, and z axis of a point of interest.

As used herein, the term “measurement” refers to 1) linear or non-linear distance calculation; 2) circumference or outline calculation from an object; 3) area calculation of a closed or semi-closed shape; or 4) visualization of location, linear or non-linear trajectories, the outline or contours of an object in two dimensional or three dimensional representations.

As used herein, the term “subject” or “patient” refers to a mammal, preferably a human, who has a medical condition that would benefit from the measuring device described herein utilized in a surgical procedure whether exploratory or corrective.

In one embodiment of the present invention there is provided a device for device for measuring an object geometry, comprising a probe having a distal contacting end and a proximal tracking end; means for tracking a location of the contacting end of the probe positioned near the proximal end electronically connected to the contacting end of the probe; and an actuation control unit electronically connected with at least the tracking sensor.

Further to this embodiment the device comprises a measurement and post processing unit in electronic communication with one or both of the tracking sensor or the actuation control unit. In these further embodiment the measurement and post processing unit comprises or is networked to a computer having a memory and a processor configured to process instructions to perform one or more functions to control activation of the device; collect data received from the contacting end of the probe and the tracking means comprising location of one or more points of interest on or proximate to the object, said location established via direct touch with the contacting end of the probe; convert three dimensional coordinates corresponding to location to two dimensional coordinates; and print or display one or both of the measurement results or image of the object geometry. Further still to these embodiments the object may be a hernia and the measurement and post processing unit are further configured to estimate hernia size and shape based on the received data; estimate a mesh size based on measured area and boundary of the hernia; and display the hernia shape and estimated mesh shape for optimization of the mesh.

In another further embodiment the device comprises means for holding the probe positioned at the proximal end of the probe. In one aspect of this further embodiment the means for holding the probe is a handle affixed to the proximal end thereof. In an alternate aspect, the handle is formed out of the proximal end of the probe. In both aspects the actuation control unit may be affixed at a distal end of the handle or, alternatively, may be affixed between the tracking means and the distal end of the handle. In yet another further embodiment the contacting end of the probe further comprises a tip affixed thereto in electronic communication with the tracking means.

In all embodiments the contacting end of the probe may be configured for direct touch measurement on the object comprising point-to-point linear or non-linear measurement, multiple points linear measurement, outlining or volume visualization. The probe may comprise a ferrous metal, a non-ferrous metal, a metal alloy, or a sterilizable plastic. Also, in all embodiments the tracking means may be affixed in direct contact with the probe or may be affixed to a spacer in direct contact with the probe. The tracking means may comprise one or more motion tracking sensors or a motion sensor array. In addition, the activation control unit may comprise one or more buttons for start, pause and start commands, one or more foot pedals, gaze- or voice-controlled activation, one or more mice or one or more keyboards. Furthermore, the device may be an endoscopic device and the object may be a hernia or other anatomic defect.

In one aspect of all embodiments, the device may be unitized such that one or both of the tracking sensor or activation control unit is permanently affixed thereto. In an alternate aspect the device may be a clip-on device such that one or both of the tracking means or activation control unit are removably affixed thereto.

In another embodiment of the present invention there is provided a method for measuring a geometry of an anatomic defect in a subject, comprising locating the anatomic defect in the subject; and measuring the geometry of the defect via direct touch with the device as described herein.

In this embodiment the measuring step may comprise directly touching, with the contacting end of the probe, one or more points of interest on or proximate to the defect in the subject; tracking the location of the one or more points as each is touched; transmitting signals corresponding to location data of the one or more points to the measuring and post processing unit; and processing the received data signals to a geometric measurement of the anatomic defect. Further to this particular embodiment the processing step comprises displaying an image of the measured geometry of the defect. In an aspect of these embodiments the anatomic defect is a hernia, and the method may further comprise sizing a mesh based on the measured geometry; and repairing the hernia with the mesh. In all embodiments the geometric measurement may correspond to a length of one or more segments of the defect, to a circumference of an edge of the defect, to an outline of the defect, to a surface area of the defect, or to a volume of the defect.

In yet another embodiment of the present invention there is provided a system for system for measuring a geometry of an object, comprising a probe having a contacting tip at a distal end and a proximal tracking end; said tip configured for direct touch of points of interest on the object; one or more tracking sensors or sensor array affixed at the proximal probe end electronically connected to the probe tip, said one or more tracking sensors or array configured to track and transmit motion of the probe tip and location of the tracking sensor(s) or array itself; a measurement and post processing unit in electronic communication with the tracking sensor or sensor array, said unit configured to process location data of the probe tip and tracking sensor(s) and to convert the data to a visual geometry of the object; and an actuation control unit electronically connected with at least the tracking sensor or the measurement and post processing unit, said control unit configured to respond to operator commands. In a further embodiment the probe may comprise a handle affixed to the proximal end thereof or that is formed out of the proximal end.

In both embodiments the actuation control unit may be affixed at a distal end of the handle or may be affixed between the one or more tracking sensors or sensor array and the distal end of the handle. In one aspect of both embodiments the probe, tracking sensor(s) or array and the actuation control unit may comprise a unitized device such that the tracking sensor(s) or array and the activation control unit are permanently affixed to the probe. In an alternate aspect the probe, one or more tracking sensors or tracking array and the actuation control unit may comprise a clip-on device such that one or both of the tracking sensor(s) or array or the activation control unit are removably affixed to the probe.

Provided herein are devices, systems and methods for the measurement of anatomic defects or geometries. The devices and methods are useful for clinical planning of surgical procedures and utilize minimally invasive and/or open direct contact of the anatomic defect or other anatomic geometry. Generally, the measurement device comprises a probe whose tip on the target side is adapted or configured to contact the points of measurement in a subject, a motion tracking system adapted or configured to track the probe's movements and a data analysis system adapted or configured to calculate the locations of the contact points of measurement and to perform post data processing. This system of measurement allows a surgeon to accurately measure the size of an anatomic defect so that appropriate treatment can be rendered.

Alternative uses for the devices, systems and methods include, but are not limited to, measurement of linear or non-linear distance between two or more points of interest without the use of a tape measure or a comparative means. Also, the devices, systems and methods are useful for measurement of a surface area or for visualization of the two dimensional geometry in a single-plane including along a curved or deformed surface of an object of interest. In addition, utility is found in the measurement or visualization of a three-dimensional volume of an object of interest. More particularly, specific clinical applications of the measuring device and system are useful in repairing hernia defects, arthoscopic prosthetic fixation, forensic measurements, bariatric and fore gut surgery and general open and closed procedures.

Particularly, the devices, systems and methods provided herein are useful in herniorraphy. Thus, the present invention provides methods of treating a hernia. The measurement device enables the surgeon to measure the size of a hernia defect so that a mesh size can be more accurately estimated than current size estimation techniques allow. This measurement involves the acquisition of a probe's tip location which makes contact with the points of interest. The device is also designed to provide measurement(s) and/or visualize the two dimensional shape(s) or three dimensional geometries of objects within real or synthetic anatomic structures which usually are not easy to perform via other measuring means, e.g., tape measure or ruler, by allowing a user to touch discretely or continuously with the probe/instrument the points included in the measurements for post data processing. Moreover, the device is designed to perform various calculations for other clinical applications which include but not limited to tailoring to target sites and fixation. The devices and systems are particularly useful in endoscopic procedures where visualization is limited.

### System Components

1. Probe is a rigid and long object where the tracking sensor is attached on. Part of the probe is introduced into the human body for the measurement and visualization. The location of the probe's tip is what usually touches specific anatomical landmarks of interest. The other side of the probe has a means for a user or operator to hold the probe, for example, a handle directly affixed to the proximal probe end or formed directly out of the proximal probe end. The probe may comprise, but is not limited to, a metal, such as a ferrous or non-ferrous metal, metal alloy or a sterilizable plastic. Metals may be a surgical stainless steel, titanium, nitinol, a silver nickel alloy, gold or gold amalgam, silicon steel, or tool steel. Plastics may be nylon, Kevlar, Teflon, Bakelite or a polycarbonate.

The probe may have, but is not limited to, a diameter about 2 mm to about 12 mm, preferably about 2 mm to about 5 mm. The length of the probe from the patient distal end to the proximal handpiece end may be about 3 cm to about 60 cm, preferably about 3 cm to about 40 cm. A representative probe has a diameter about 5 mm and a length of about 40 cm. Dimensions may be determined based on the use for the measuring device comprising the probe. The probe tip may have various useful shapes. For example, the probe tip may be straight, curved or angled, may be an articulating tip or other variable tip design which can be affixed as needed.

In a non-limiting example the probe may be a 40 cm insertion rod with a tip with variable geometries at the distal or patient end and with a handle attached to the proximal end of the probe. A tracking sensor may be located or attached at or between the point where the insertion rod is connected to the handle. A control unit may comprise buttons affixed on the handle nearer the patient end of the probe in electronic communication with the probe tip and/or tracking sensor.

2. A tracking means, for example, one or more tracking sensors or an array of tracking markers which is used to measure the location information of this tracking sensor when used with various kinds of motion tracking systems. The measuring device is designed to utilize various tracking technologies known in the art. The tracking sensor(s) may be active or passive sensors. The tracking sensor is attached temporarily or permanently to the probe to calculate the location information of the probe tip using any existing motion tracking technologies, including, but not limited to, passive and active optical, electromagnetic, radiofrequency, ultrasound, accelerometer. gyroscopic, or video image based motion tracking systems.

3. Control unit is an actuation device unit which works with the tracking sensor and/or the measurement & post processing unit. The control unit includes, but is not limited to, buttons on device, buttons on measurement & post processing unit, foot pedals, gesture recognition systems, gaze control systems, keyboards, mouse.

4. Measurement and post processing unit is a device unit or an array of multiple device units which measure the location of the tracking sensor, calculate the location of the probe tip, calculate various kinds of distances from the probe tip's movement trajectories, visualize the probe tip's movement trajectories, and perform post processing of probe tip movement data to provide additional information which includes, but is not limited to, the area of the circumference of an anatomical object and the size of the Hernia defect. This unit comprises networkable computer(s) with memories and processors effective to receive data and execute instructions to process data received from the probe, monitors, data delivery and processing hardware and software that, as is known in the art may be wired or wireless, hand-held or table top.

5. Power specifications: The system may use an AC electric power supply, may be wirelessly battery operated or may use inductance technologies to provide power.

### Hardware and Software

1. Activation Control:

The system provides various options for operators to start (activate) and stop (deactivate) the data collection process of the probe tip. An operator can i) manipulate a button on the probe to start and stop the data collection process; ii) manipulate a foot pedal to start and stop the data collection process; iii) use voice activation by speaking commands such as “Start”, “Pause”, and “Stop” to start and stop the data collection process; iv) use gaze controlled activation via utilization of a pointer system that includes a gaze control where the user looks in a direction or at an image to control the system; and v) use gesture controlled activation via utilization of a pointer system that includes a gesture recognition system which provides some control for the system.

2. Data Collection:

For location data of the probe tip the software defines a global coordinate system with a center position whose coordinate is (0, 0, 0) in (x, y, z) notation. The software identifies the location (in x, y, z coordinates) of the probe tip in three dimensional space and records its coordinates in real time while the data collection mode is active. In Continuous mode, the software records the location data until the operator deactivates data collection mode. by pressing the data button or releasing the pedal or saying “Pause” or “Stop”. The software provides a continuous auditory tone as a confirmation of having the location data being collected. In Discrete mode, the software records the instantaneous location data when the operator activates data collection mode by pressing and holding the data button for two seconds, pushing the pedal for two seconds or saying “Point”. The software provides a short auditory tone for confirmation of data collection.

3. Three Dimensional to Two Dimensional Coordinate Conversion:

The software calculates the middle point of the multiple locations obtained from the hernia defect. The software converts three dimensional coordinates of the multiple location data into two dimensional coordinates by projecting these multiple locations into a plane (defined by two lines) which yields the maximum distances between the middle point and each of multiple locations. The software outputs two dimensional coordinates of the multiple locations.

4. Specific Application—Hernia Size and Shape Estimation:

For estimating hernia size and shape functions, the software allows the operator to choose between oval or specific shape approximation for the estimation of mesh shape. In oval mode the software creates an oval which covers 95% of multiple location data obtained from the targeted hernia defect by using mathematical algorithm such as Principal Component Analysis (PCA). In specific mode the software creates a boundary profile by connecting adjacent location data.

For estimating mesh size functions, the software allows the operator to estimate the mesh size by using area and boundary methods. For area (%), the software accepts a numeric input of the percentage of the mesh area (e.g. 150%) to estimate the mesh size. For boundary (cm), the software accepts a numeric input for how far the boundary of the mesh will be apart from the boundary of the simplified hernia geometry.

For size adjustment functions, the software displays the hernia shape and estimated mesh shape and size for the operator's review. The software allows the operator to adjust the size of estimated mesh for the optimal mesh setting.

For printing functions, the software provides the operator with estimated size and shape for mesh cutting.

### Probe System Types

1. A unitized probe system is a probe with the tracking sensor and/or control unit permanently attached onto the probe.

2. A clip-on probe system comprises a tracking sensor and/or control unit that is clipped onto a probe which function like the unitized probe system. The clip-on is affixed via a clamp or clip which is designed to keep the system and instrument connected as a single unit and, if needed, in a specific orientation. It is necessary to ensure that the function of the “host” probe is not affected by the “symbiotic” clamp/clip system.

### Usage Modes

1. Point to point linear measurement: The linear measurement between two points defined as “Start” and “Stop” locations acquired by having the probe tip touching at the two points.

2. Point to point non-linear measurement: The non-linear measurement between two points, defined as “Start” and “Stop” locations, acquired by having the probe tip continuously traveling from the start location to stop location. The final measurement is obtained from the continuous trajectory of the tip movement of the probe.

3. Multiple points linear measurement: This is similar to the point to point linear measurement except that between the start and stop locations, there are multiple “Via point(s)”. The probe tip discretely touches each point to identify the locations of each and ultimately generates multiple line segments. The partial measurements take place between two successive points and then added up to create the final measurement between the start and the stop locations with multiples segments.

4. Outlining: For this mode, the probe tip moves along the circumference or outline of an object and the traveling trajectory, discrete or continuous, of the probe tip is measured. Distance, shape, or area of the outline can be the outcome of this measurement.

5. Volume visualization: In addition to outlining an object, the probe tip travels continuously in a single or multiple sessions on the surface of the object to visualize the three dimensional contour and the shape of the object.

As described below, the invention provides a number of surgical advantages and uses, however such advantages and uses are not limited by such description. Embodiments of the present invention are better illustrated with reference to the Figure(s), however, such reference is not meant to limit the present invention in any fashion. The embodiments and variations described in detail herein are to be interpreted by the appended claims and equivalents thereof.

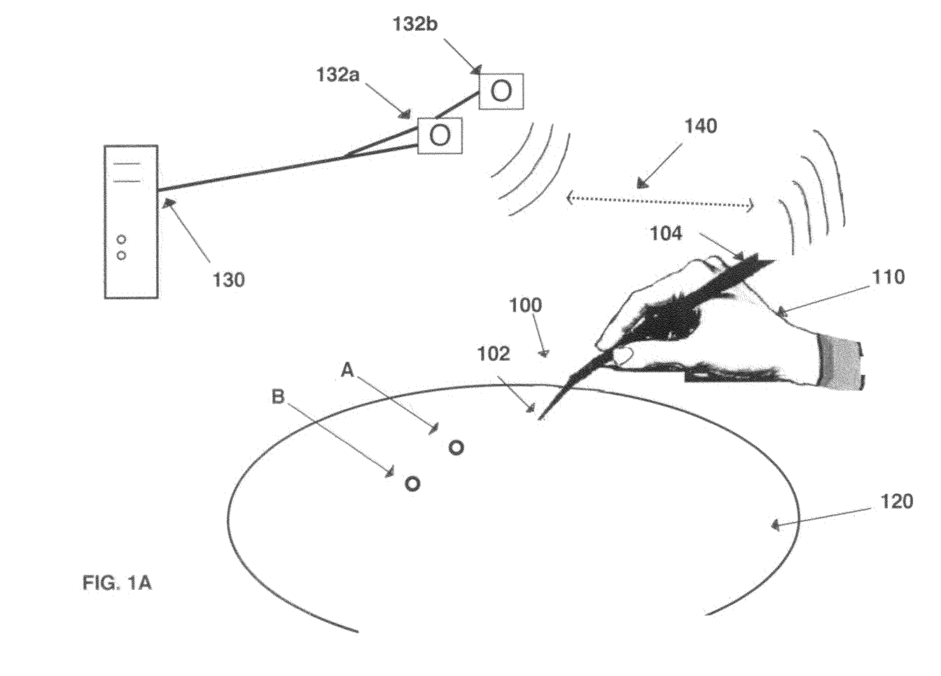

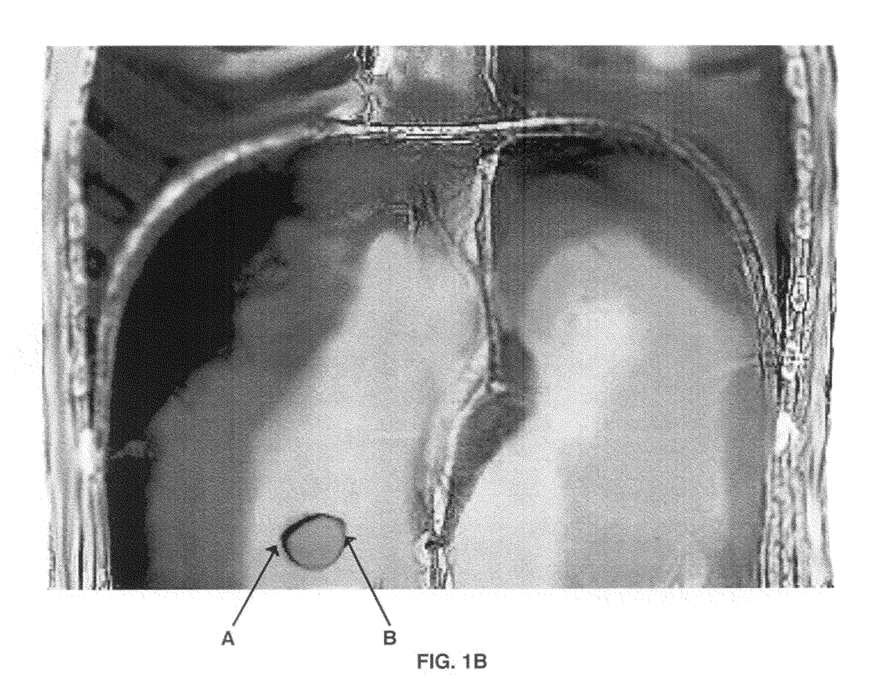

FIG. 1A depicts a probe 100 held by a user 110 to identify an area of interest between points A and B in an anatomic cavity 120. The probe has a distal contact end 102 adapted to touch individually or traverse between points A and B and a proximal tracking end 104 electronically connected to a tracking system 130 configured for either passive or active motion tracking techniques depending on the use and environment. Alternatively, tracking systems such as accelerometer/gyroscopic systems embedded in the probe can also provide location information. Once in place the probe is used to touch or traverse points A and/or B or an area of interest encompassing the same. The tracking system locates the distal contact end, via direct or indirect transformation of tracking signals 140 emitted by the proximal tracking end at transformers 132a,b. A computational system comprising the tracking system or electronically linked thereto determines the distances, areas, and geometries comprising points A and B. FIG. 1B depicts a hernia, as an example of an anatomic defect, showing points of interest A and B, as in FIG. 1A, on the edge of a hernia on the abdominal wall. A user probe would touch the points A and B using the probe 100 and determine a measurement from point A to point B or a total measurement around the defect, if the probe was moved around the edge of the hernia.

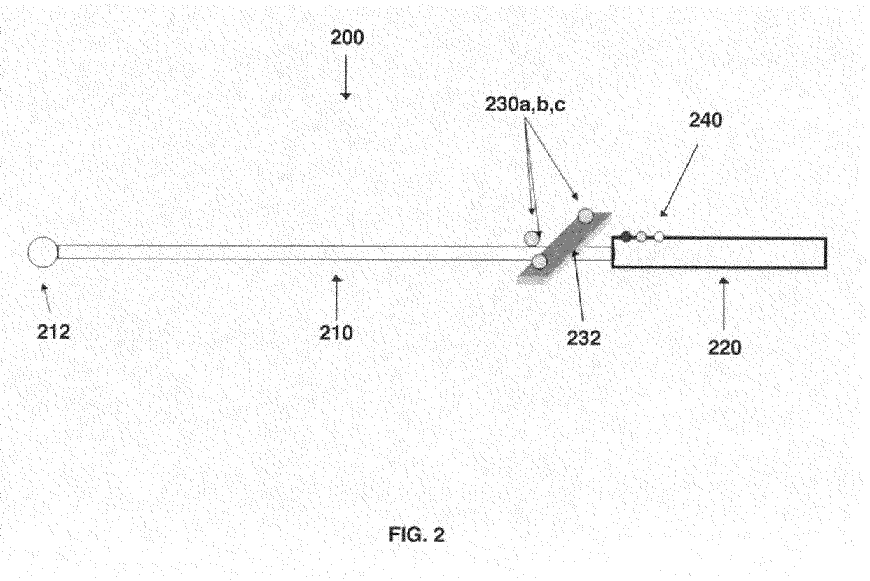

FIG. 2 depicts a unitized measuring device 200. The device comprises a rod-like probe 210 of about 65 cm in length. It is noted that the rod may be any length and be constructed of one or more materials as described herein. The distal end of the probe may be softened by standard methods to be atraumatic for a patient. Optionally, the distal end of the rod comprises a tip 212 attached via an attaching mechanism, such as, but not limited to a screw, to provide for variability in the tip design and geometry. The device has a handle 220 at the proximal end of the probe that is either affixed thereto, or, alternatively, the proximal end of the probe is formed or shaped as a handle.

The device comprises a tracking array or plurality of tracking sensors or markers 230a,b,c, although other tracking sensors or arrays as described herein are utilizable with the device. As the probe is described as 65 cm in length, at a point 232 that is about 45 cm from the tip, an optical tracking array, magnetic sensor, accelerometer, or other tracking device is connected, fastened or affixed at its center to the rod. Optionally, a mount or spacer is positioned between the probe and the tracking sensor or array. For calibration purposes (see Example 1) the exact distance from the tip to the point of center contact of the tracker sensor or array to the probe must be determined.

The handle 220 comprises a button or an array of buttons 240 electronically integrated within the handle at its distal end as controls for the device. The button(s) or button array may be integrated with the probe tip and tracking sensor/array with a wire or wirelessly as is known in the art. The button(s) or button array are configured or adapted to send input to the tracking system, such as system 130 in FIG. 1A. The button(s) are configured so that on/off is controlled from this point by either single push or some combination of on/off, discreet/continuous mode, or mesh on off. As described herein alternate control methods include, but are not limited to, a foot pedal with similar specifications as the buttons, voice control, eye tracking control, or other means. In another option a visible indicator may be mounted at a point 250 near the handle configured to indicate system status, for example, but not limited to, on, off, data acquisition in progress, or failure. The indicator may be wired to the device or may monitor the device wirelessly.

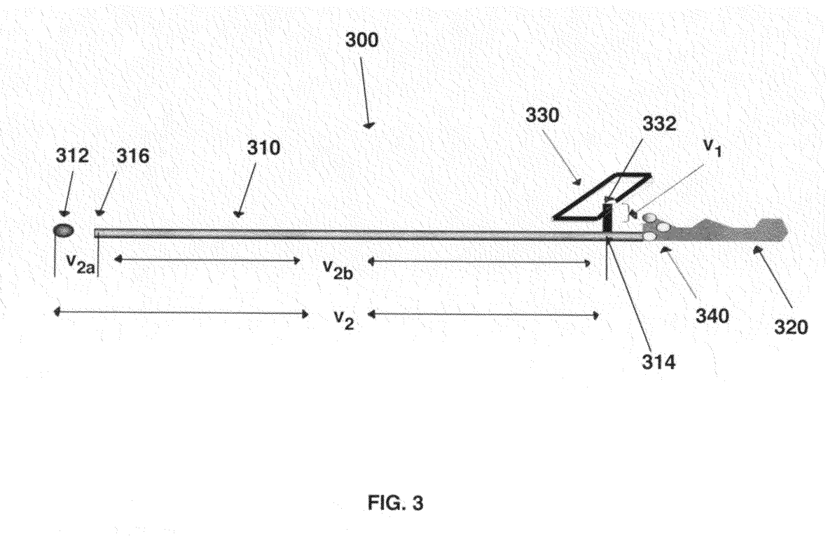

FIG. 3 depicts another unitized device 300 showing vectors necessary for probe calibration (see Example 1). The device comprises a rod-like probe 310 with a tip 312 at the distal end and a handle 320 affixed at the proximal end of the probe. A tracking sensor 330 is mounted to the probe with a spacer 332. As described in FIG. 2, the handle has a control unit 340 comprising a set of buttons integrated at the distal end thereof. Vector₁ V₁ is from the center of sensor coordinate 332 to the attachment center 314. Vector₂ V₂ is the sum of the vector distance V₂ₐ between the distal end of the probe and the probe tip attached thereto and the vector distance V_{2b} between the attachment center and the distal end of the probe 316.

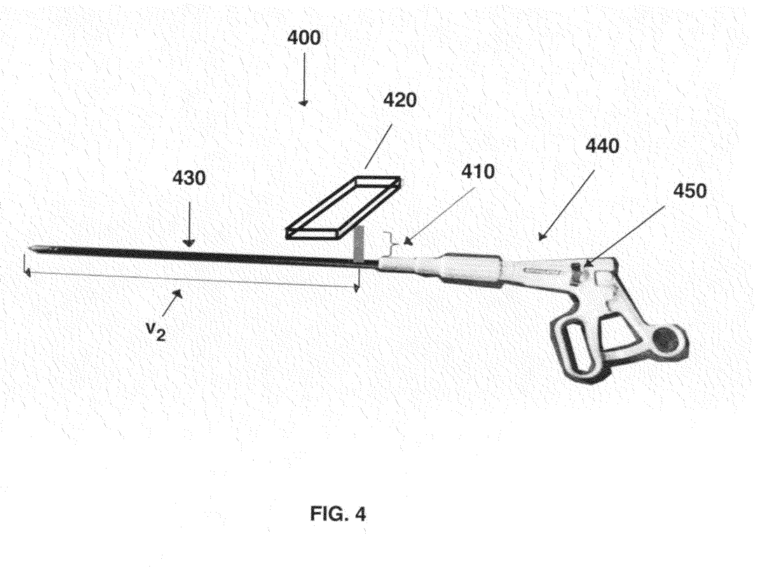

FIG. 4 depicts a clip-on endoscopic device 400 for endoscopic surgical procedures. A clip 410 configured to accept a tracking array/sensor 420 is mounted on the probe component 430 of the endoscopic device as close as possible to the handle component 440. As in FIG. 2, one or more buttons or button array are electronically integrated into the endoscope at the hand side 450 of the clip via wire or wireless integration for user accessibility while the endoscope is being held. The button(s) or button array are configured as for FIG. 2. Also, as in FIG. 2, a wired or wireless visible indicator 460 may be mounted near the patient side of the clip in a visible fashion which would indicate system status. Corresponding to FIG. 3 the exact distance vector V₂ from the tip to the point of center contact of the tracker/sensor must be determined.

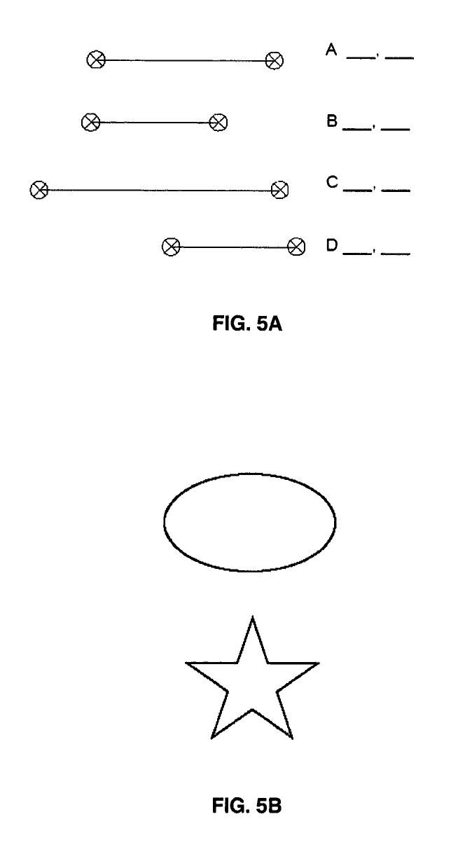

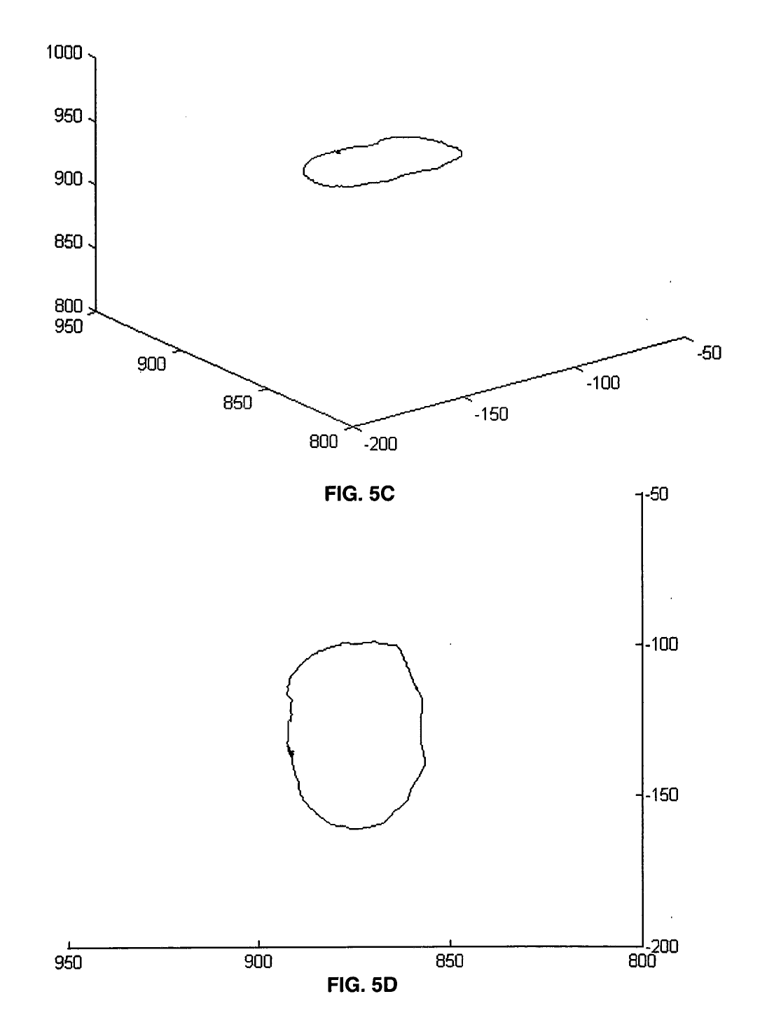

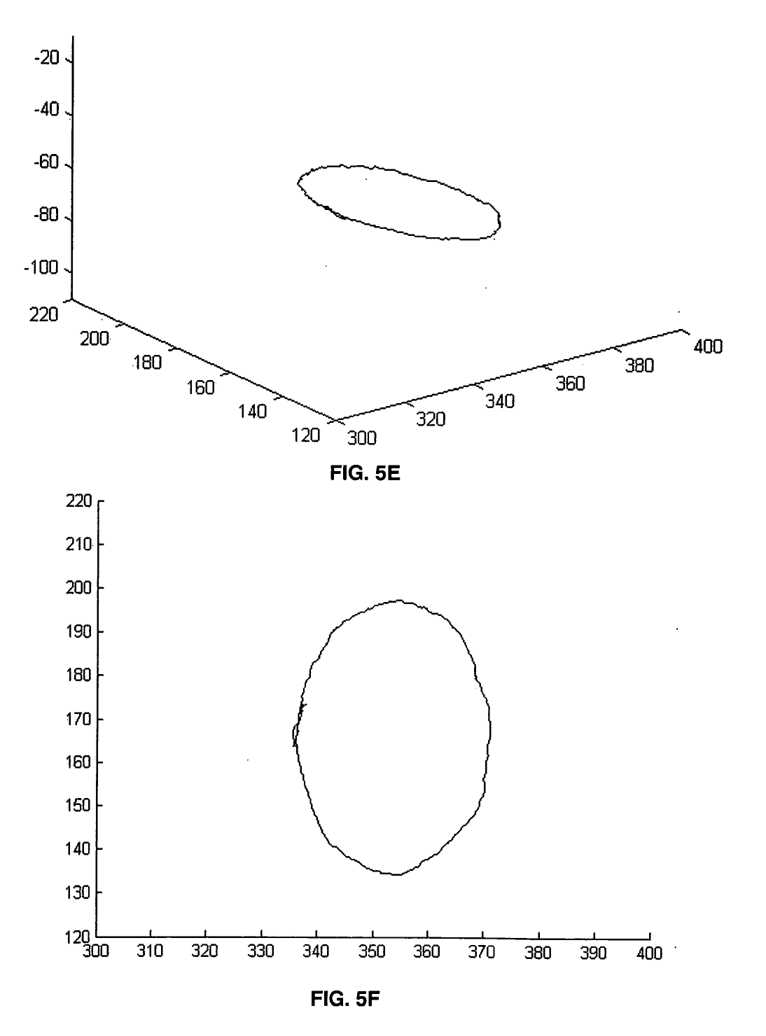

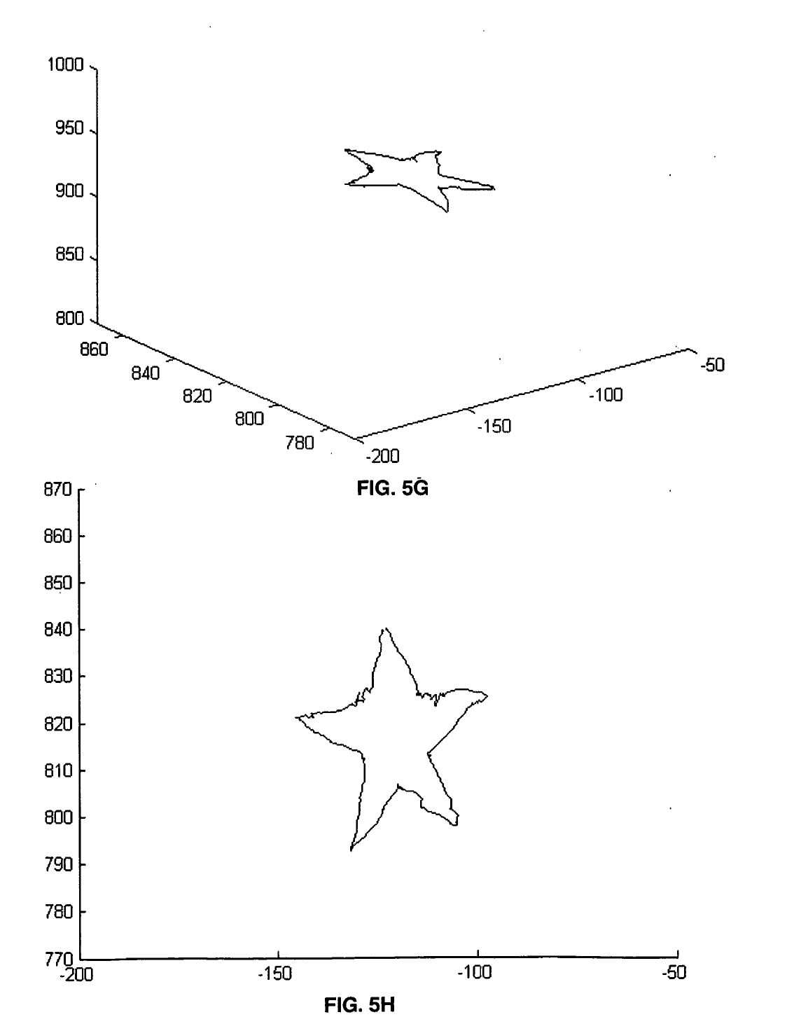

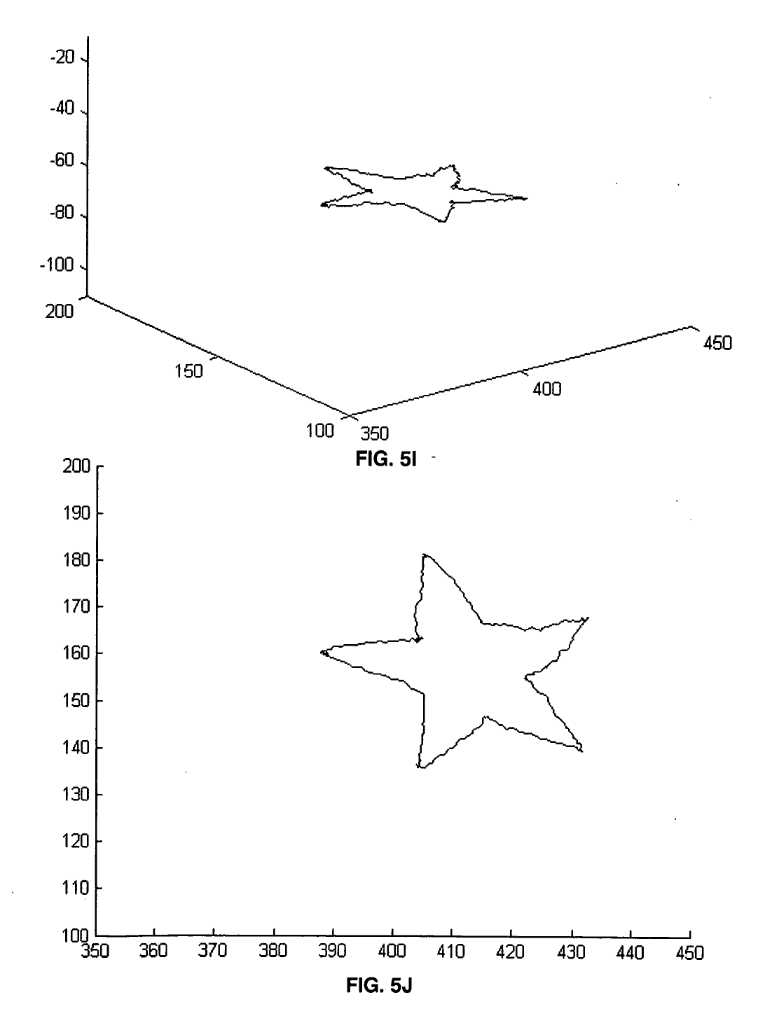

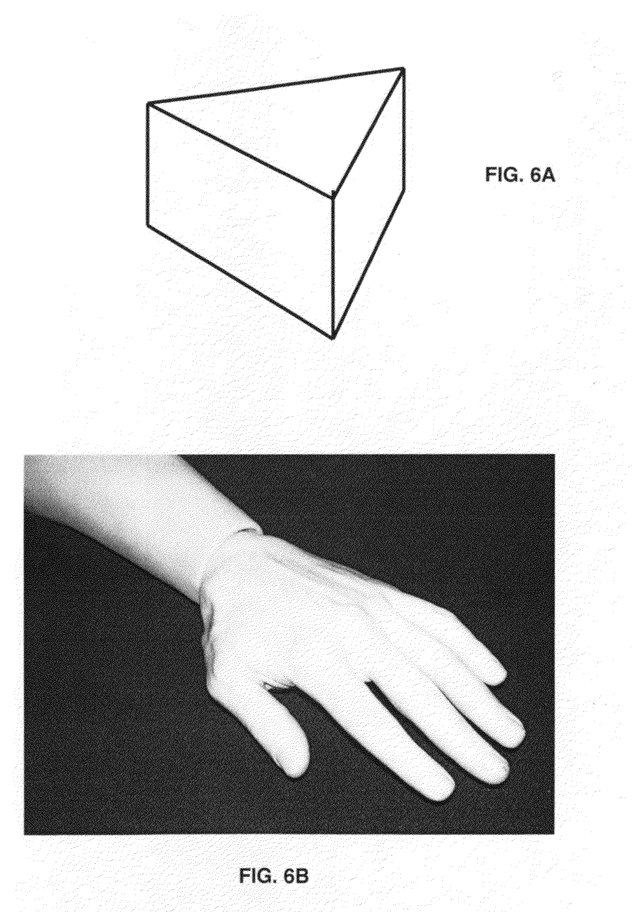

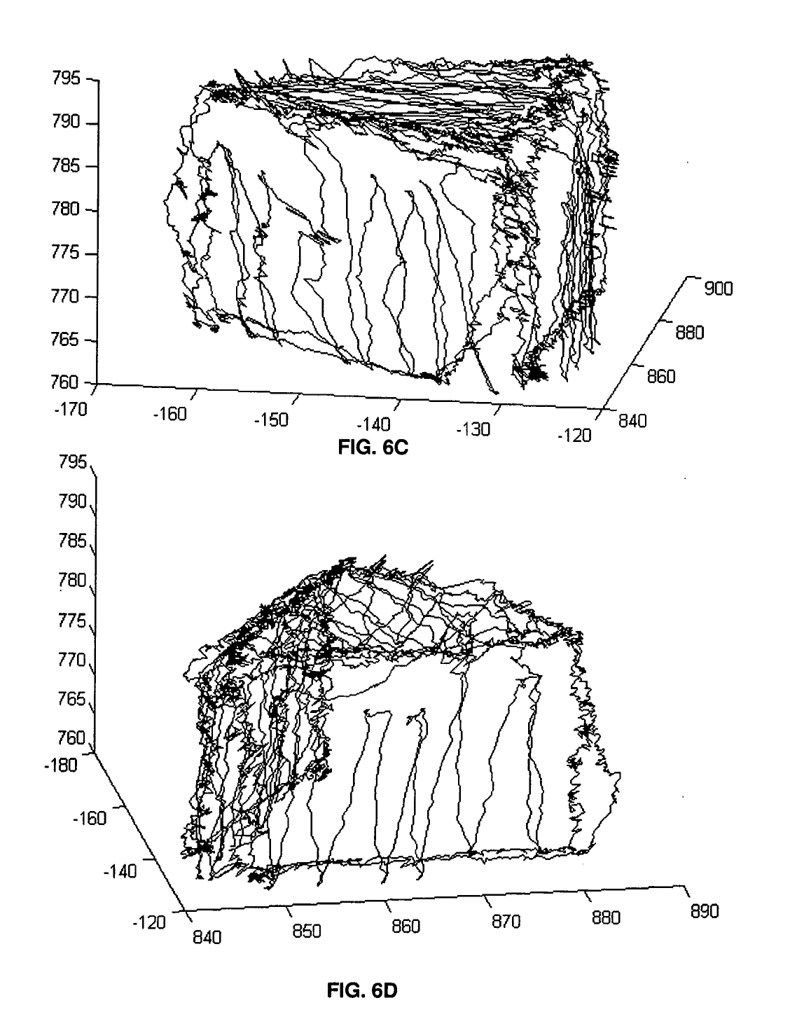

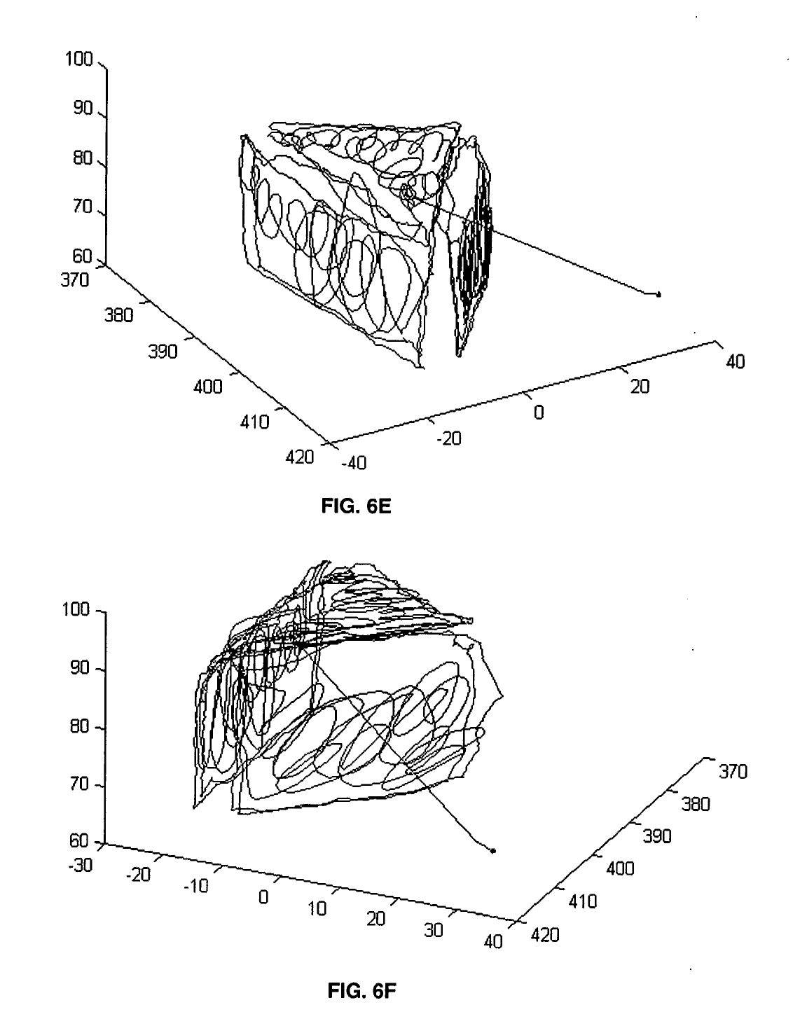

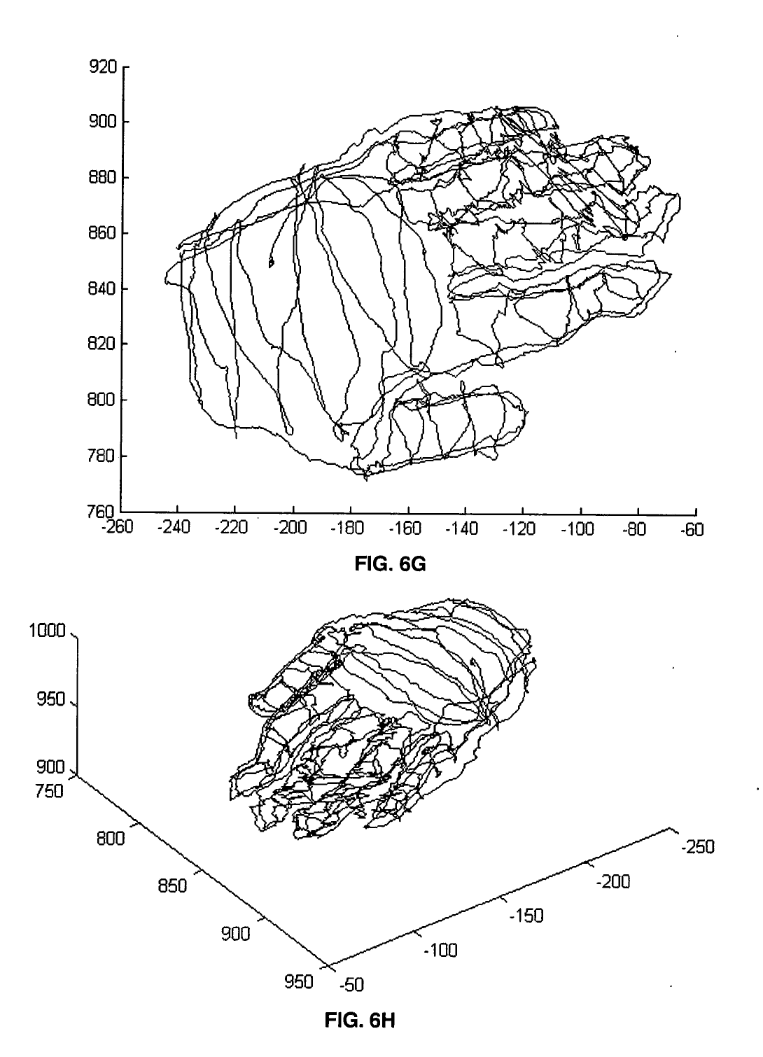

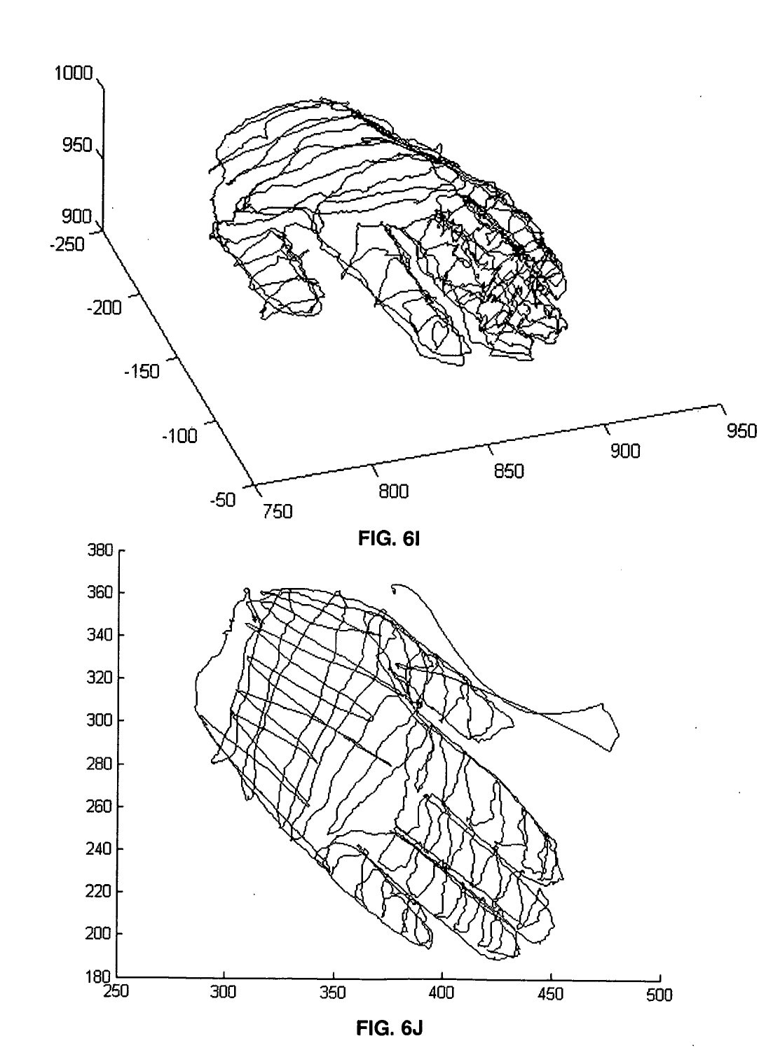

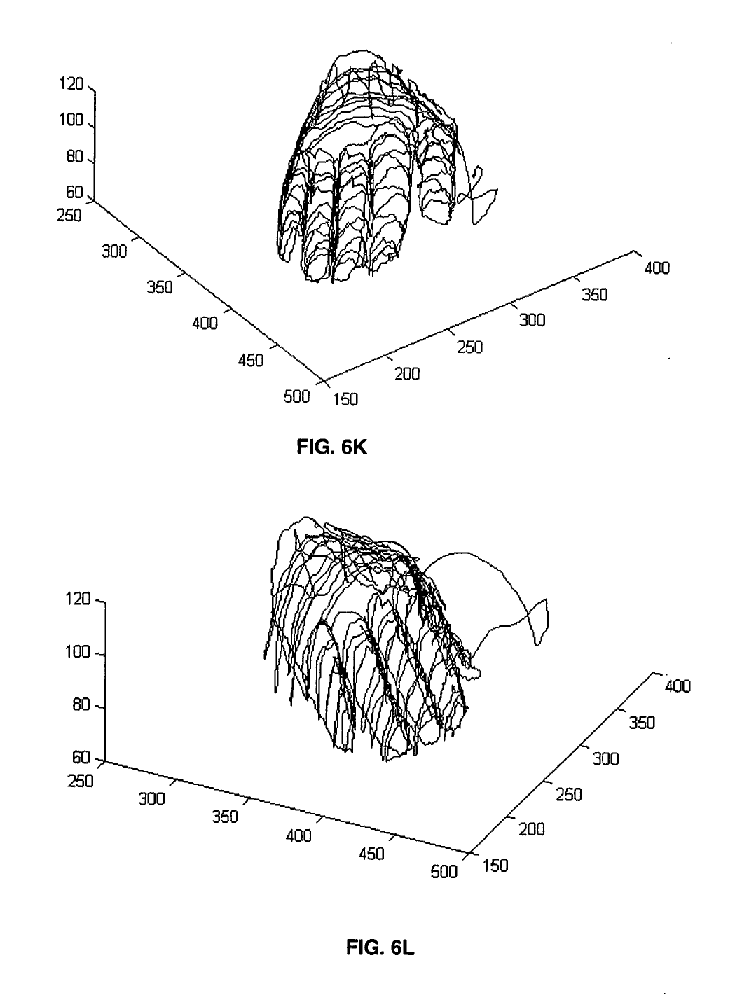

FIGS. 5A-5B depict line segments of various lengths and an oval and star shaped figures to evaluate the accuracy of a probe with an optical or electromagnetic tip and tracking sensor configuration for two-dimensional measurement and/or tracing. FIGS. 5C-5D show the results of tracing the oval outline with an optical tip/sensor configuration and FIGS. 5E-5F show the results of tracing the oval outline with an electromagnetic tip/sensor configuration both as a three-dimensional (x,y,z) plot or as a two-dimensional (x,y) plot, respectively. FIGS. 5G-5H show the corresponding three- and two-dimensional plots for tracing the star outline using the optical probe and FIGS. 5I-5J show the corresponding three- and two-dimensional plots for the star using the electromagnetic probe. FIGS. 6A-6B depict a solid three-dimensional triangular object and a mannequin's hand to evaluate the accuracy of the optical and electromagnetic probes in FIGS. 5A-5J for surface tracing. FIGS. 6C-6D show three surfaces of the triangular object traced using the optical probe in different orientations as a three-dimensional (x,y,z) plot. FIGS. 6E-6F show the triangle surfaces traced using the electromagnetic probe. FIGS. 6G-6I show surface tracing of the back of the hand as a two-dimensional plot and the palm and back of the hand as three-dimensional (x,y,z) plots obtained from the optical probe. FIGS. 6J-6L show surface tracing of the palm of the hand as a two-dimensional (x,y) plot and of the back of the hand in different orientations as three-dimensional (x,y,z) plot using the electromagnetic sensor.

The following examples are given for the purpose of illustrating various embodiments of the invention and are not meant to limit the present invention in any fashion.

### Example 1

Measurement with the Probe

### Probe Calibration

This calibration process is required for all possible motion tracking technologies. With most motion tracking systems, it is physically impossible to attach the tracking sensor at the tip of probe. With some motion tracking technologies such as electromagnetic motion tracking, the sensor may be attached to the tip of the probe but this creates another issue in sterilization of the sensors. To avoid these problems, the motion sensor is placed at the proximal position, that is, near the handle of the probe. Then a calibration process is required to define the relative location (x, y, z in three dimensional space) of the tip of probe with reference to the coordinate center defined at the location of tracking sensor.

The coordinate center for each tracking sensors are defined according to the type of tracking utilized. In optical tracking with an array of reflective markers the coordinate center is the physical center location of the multiple markers. In electromagnetic tracking or if using an accelerometer or in other tracking technologies, the manufacturer's design is followed in these instances where the coordinate center is usually physical center of the sensor. Examples may include but not limited to articulated arms for which known paths and finite locations exist).

Common dimensional information required for the calibration process are a first and second distance measurement. Distance 1 is the orthogonal distance between the center of sensor coordinate to the point where orthogonal projection of the center of sensor coordinate meet with the line in the middle of the probe along the long axis of the probe (attachment center, FIG. 3). Distance 2 is the distance between the tip of the probe and the attachment center point (FIG. 3).

Two vectors can be created from these measurements. Vector₁ V₁, is a vector from the center of sensor coordinate to the attachment center. Vector₂, V₂, is a vector from the attachment center to the tip of the probe and is the sum of V₂ₐ, distal end of probe to end of probe tip and V_{2b}, attachment center to distal end of probe. From these two vectors, the motion tracking system will create a resultant vector (V_{R}, not shown) from the center of sensor coordinate to the tip of the probe. This resultant vector is used to identify the location of the tip with reference to the sensor coordinate which represents the instrument handle movement.

For the unitized probe the pre-measured dimensions for the vector calculation process described above are available in the system and if an identifier is pre-assigned to each permanent probe, then the identifier will be entered to the system so be calibrated. For the clip on probe system, the two distances are directly measured and then typed into the system to calculate the resultant vector so that the system can ultimately be calculated the location of the tip. It is also possible that this distance could be entered into the systems via CAD diagrams, part number, which would reference geometric specifications of an existing instrument or analog physical measurement.

### Example 2

### Operation Procedures for Measurement

### General Overview

The system power is turned on. The probe is then introduced into the body to verify whether the tip of the probe can reach target. A continuous or discrete mode is selected from a control panel. A system/data acquitsion or start is initiated via, for example, a mouse, a foot pedal, a voice activation system, and/or gestured controlled system. Data collection begins, i.e., linear or point-to-point.

### Discreet Mode: Line Segment

The operator moves the probe tip to a point of interest (initial point) and then activates data collection mode is activated (options: holding the data button, pushing a pedal switch, or saying “Start”). Two seconds after activating the data collection mode, the operator gets a confirmation, audible and/or visible, e,g., hears a continuous auditory tone which confirms that the data is being collected. The operator then moves to the final point of interest and activates data collection mode (options: holding the data button, pushing a pedal switch, or saying “Start”). Two seconds after activating the data collection mode, the operator gets a confirmation, audible and/or visible, e,g., hears a continuous auditory tone which confirms that the data is being collected. The system will then via a “monitor” or via audio return to the user the length of the segment acquired.

### Discreet Mode: Non-Linear or Segmented Mode

The operator moves the probe tip to a point of interest (initial point) and then activates data collection mode is activated. Optionally, the operator may hold the data button, push a pedal switch, or saying “Start”. Two seconds after activating the data collection mode, the operator gets an audible or visible confirmation, e.g., hears a continuous auditory tone which confirms that the data is being collected. The operator then moves to the final point of interest and activates data collection mode is activated, as described. Two seconds after activating the data collection mode, the operator gets audible or visible confirmation, as described. The operator then moves to another point, and activates data collection, and then continues to do so until the total number of segments is collected. The operator activates a “measurement summation” function which will total the segments. The system then, via a “monitor” or via audio, return to the user the length of the segment acquired. Note this feature could be used to “run” bowel and measure at the same time.

### Continuous Mode

The operator moves the probe tip along the circumference of the targeted hernia defect while data collection mode is activated as described for discrete mode. Two seconds after activating the data collection mode, the operator gets an audible and/or visible confirmation, as described. If the operator needs to move the probe tip without recording the location data, the operator temporarily pauses the data collection by, optionally, releasing the data button, releasing the pedal switch, or saying “Pause”. Once the operator places to the probe tip to desired location, the operator resumes the data collection, as described for previous steps. Once the operator reaches at where the data collection started or the location where the operator wants to stop data collection, the operator deactivates data collection mode, as described for previous deactivation steps.

### Discrete Mode: Circumference

The operator moves the probe tip to the first desired location on the circumference of the targeted hernia defect. Once the probe tip is at the desired location, the operator presses and holds the data button for two seconds, pushes the pedal for two seconds or says “Point”. Once the three dimensional data of the location is measured, the operator hears a short auditory tone for confirmation of data collection. The operator moves to the next desired point on the circumference of the targeted hernia defect and then repeat the above step. The operator repeats the above two steps until the operator obtained the location data for the desired number of points. The operator deactivates data collection mode, as described in above steps. A system/process “Stop” is initiated to terminate the data collection process

### Example 3

### Measuring a Hernia Defect

The calibration and operation procedures for measuring a hernia defect in discrete mode, utilizing point-to-point, linear or non-linear, circumferential, or continuous data acquisition methods are substantially identical to the operation of the device as described in Examples 1 and 2. In a discrete mode for circumference measurement of the defect, after data collection is terminated, the system initiates a process called “Hernia Estimation”.

In “Hernia Estimation” the system displays simplified two-dimensional geometry of the hernia defect and the operator responds to the question “Will you accept this estimation (Yes/No). If the operator selects “No”, the operator will repeat the above steps until the operator obtains an acceptable estimation. If the operator selects “Yes”, the system will display a message “Estimation accepted”.

The operator selects options for the estimation of mesh shape (Oval/Specific). For an oval, the system creates an oval which covers 95% of the continuous or discrete location data of the targeted hernia defect. For a specific mesh shape the system creates a curved shape which covers 95% of the continuous or discrete location data of the targeted hernia defect. The system then displays the simplified hernia geometry as well as the boundaries of the oval or the curved shape.

The operator then selects options for the estimation of mesh size (Area/Boundary). For area (%), the operator enters the percentage of the mesh size (e.g. 150%) to estimate the mesh size. For boundary (cm), the area enters how far the boundary of the mesh will be apart from the boundary of the simplified hernia geometry. The system displays the simplified hernia geometry and the estimated mesh cutting based on the selections made above. The operator uses “+” and “-” buttons to increase or decreased the size of the estimated mesh cutting for adjustment of the size. after making this adjustment, the operator pushes the button “Finish”. The system provides the operator with estimated size and shape for mesh cutting.

### Example 4

Tracing Shapes and Objects with Optical and Electromagnetic Tracking

Measurements are made with an infrared optical motion tracking system (Vicon) with a probe using a tracking sensor comprising a cluster marker of retro-reflective markers to create a virtual marker to track the tip location and with an electromagnetic tracking system (Ascension Technology) where the probe comprises an electromagnetic sensor in the probe tip.

### Line Length Measurement and Oval and Star Shape Tracing

FIGS. 5A-5B depict the two-dimensional templates for measuring line segments and tracing the boundaries of an oval and a star. Table 1 compares the measurements obtained using optical tracking and calipers.

TABLE 1

| | | Line A | Line B | Line C | Line D |
|---|---|---|---|---|---|
| | | 65.0 mm | 46.5 mm | 88.5 mm | 46.0 mm |
| Optical | Trial 1 | 65.1 | 45.8 | 86.3 | 44.7 |
| | Trial 2 | 65.5 | 45.8 | 85.0 | 42.2 |
| Electro- | Trial 1 | 62.3 | 45.2 | 86.6 | 44.7 |
| magnetic | | | | | |

The results from tracing the oval (FIGS. 5C-5F) and the star (FIGS. 5G-5J) using the optical (FIGS. 5C-5D, 5G-5H) and the electromagnetic (FIGS. 5E-5F, 5I-5J) probes are depicted in two- and three-dimensional spaces.

### Surface Tracing

FIGS. 6A-6B are images of a solid triangular shaped object and a mannequin's left hand, the surfaces of which are traced by the optical and electromagnetic probes. FIGS. 6C-6F depict a tracing of three surfaces of the triangular object. The optical system provides a slightly better three-dimensional rendering compared to the electromagnetic device, although all views are identifiable. FIGS. 6G-6L depict tracings of the back and the palm of the left hand. The fingers are clearly rendered and whether the hand is palm up or down is easily determined for both the optical and the electromagnetic tip/tracking sensor devices.

Any patents or publications mentioned in this specification are indicative of the levels of those skilled in the art to which the invention pertains. These patents and publications are herein incorporated by reference to the same extent as if each individual publication was incorporated specifically and individually by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A device for measuring an object geometry, comprising:
a probe having a distal contacting end and a proximal tracking end;
means for tracking a location of the contacting end of the probe positioned near the proximal end electronically connected to the contacting end of the probe; and
an actuation control unit electronically connected with at least the tracking sensor.

2. The measuring device of claim 1, further comprising:
a measurement and post processing unit in electronic communication with one or both of the tracking sensor or the actuation control unit, said measurement and post processing comprising or is networked to a computer having a memory and a processor configured to process instructions to:
control activation of the device;
collect data received from the contacting end of the probe and the tracking means comprising location of one or more points of interest on or proximate to the object, said location established via direct touch with the contacting end of the probe;
convert three dimensional coordinates corresponding to location to two dimensional coordinates; and
print or display one or both of the measurement results or image of the object geometry.

3. The measuring device of claim 1, wherein the object is a hernia, the measurement and post processing unit further configured to:
estimate hernia size and shape based on the received data;
estimate a mesh size based on measured area and boundary of the hernia; and
display the hernia shape and estimated mesh shape for optimization of the mesh.

4. The measuring device of claim 1, further comprising:
a handle affixed to the proximal end thereof or is formed out of the proximal end of the probe.

5. The measuring device of claim 4, wherein the actuation control unit is affixed at a distal end of the handle or between the tracking means and the distal end of the handle.

6. The measuring device of claim 1, wherein the contacting end of the probe further comprises a tip affixed thereto in electronic communication with the tracking means.

7. The measuring device of claim 1, wherein the contacting end of the probe is configured for direct touch measurement on the object comprising point-to-point linear or non-linear measurement, multiple points linear measurement, outlining or volume visualization.

8. The measuring device of claim 1, wherein the tracking means is affixed in direct contact with the probe or is affixed to a spacer in direct contact with the probe.

9. The measuring device of claim 1, wherein the tracking means comprises one or more motion tracking sensors or a motion sensor array.

10. The measuring device of claim 1, wherein the activation control unit comprises one or more buttons for start, pause and start commands, one or more foot pedals, gaze- or voice-controlled activation, one or more mice or one or more keyboards.

11. The measuring device of claim 1, wherein the device is unitized such that one or both of the tracking sensor or activation control unit is permanently affixed thereto or is a clip-on device such that one or both of the tracking means or activation control unit are removably affixed thereto.

12. The measuring device of claim 1, wherein the device is an endoscopic device.

13. The measuring device of claim 1, wherein the object is a hernia or other anatomic defect.

14. A method for measuring a geometry of an anatomic defect in a subject, comprising:
locating the anatomic defect in the subject; and
measuring the geometry of the defect via direct touch with the device of claim 1.

15. The method of claim 14, wherein the measuring step comprises:
directly touching, with the contacting end of the probe, one or more points of interest on or proximate to the defect in the subject;
tracking the location of the one or more points as each is touched;
transmitting signals corresponding to location data of the one or more points to the measuring and post processing unit; and
processing the received data signals to a geometric measurement of the anatomic defect.

16. The method of claim 15, wherein the geometric measurement corresponds to a length of one or more segments of the defect, to a circumference of an edge of the defect, to an outline of the defect, to a surface area of the defect, or to a volume of the defect.

17. The method of claim 14, wherein the processing step further comprises displaying an image of the measured geometry of the defect.

18. The method of claim 14, wherein the anatomic defect is a hernia, the method further comprising:
sizing a mesh based on the measured geometry; and
repairing the hernia with the mesh.

19. A system for measuring a geometry of an anatomic object, comprising:
a probe having a contacting tip at a distal end and a proximal tracking end;
said tip configured for direct touch of points of interest on the object;
one or more tracking sensors or sensor array affixed at the proximal probe end electronically connected to the probe tip, said one or more tracking sensors or array configured to track and transmit motion of the probe tip and location of the tracking sensor(s) or array itself;
a measurement and post processing unit in electronic communication with the tracking sensor or sensor array, said unit configured to process location data of the probe tip and tracking sensor(s) and to convert the data to a visual geometry of the object; and
an actuation control unit electronically connected with at least the tracking sensor or the measurement and post processing unit, said control unit configured to respond to operator commands.

20. The object measuring system of claim 19, wherein the probe further comprises a handle affixed to the proximal end thereof or is formed out of the proximal end.

21. The object measuring system of claim 19, wherein the actuation control unit is affixed at a distal end of the handle or between the one or more tracking sensors or sensor array and the distal end of the handle.

22. The object measuring system of claim 19, wherein the probe, one or more tracking sensor(s) or array and the actuation control unit comprise a unitized device such that the tracking sensor(s) or array and the activation control unit are permanently affixed to the probe or comprises a clip-on device such that one or both of the tracking sensor(s) or array or the activation control unit are removably affixed to the probe.
